(19) United States
(12) Patent Application Publication
(10) Pub. No.: US 2013/0211201 A1
(43) Pub. Date: Aug. 15, 2013

(54) THE SURGICAL VISUAL FIELD ENHANCER APPARATUS AND ITS METHOD OF USE

(71) Applicant: Sunton Wongsiri, Hatyai (TH)

(72) Inventor: Sunton Wongsiri, Hatyai (TH)

(73) Assignee: SURGICAL INNOVATION HEALTHCARE CO., LTD., Bangkok (TH)

(21) Appl. No.: 13/522,215

(22) PCT Filed: Jan. 14, 2010
(86) PCT No.: PCT/TH10/00002

§ 371 Date: Nov. 16, 2012

(51) Int. Cl. A61B 1/32; A61B 1/06

(52) U.S. Cl. CPC A61B 1/32; A61B 1/06
USPC 600/202; 600/201; 600/203; 600/245

(57) ABSTRACT

The Surgical Visual Field Enhancer is used to improve the visual field under a limited skin incision. It includes various parts to make the surgeon's work easier, such as the removable handle, the tunnel opening guide, the tunnel dilator, the lighting tool and the lighting transmitter. Initially, the user holds the surgical tool body of the surgical visual field enhancer and uses the tunnel opening end to create a tunnel through the soft tissue. The tunnel opening end is composed of the tunnel opening tip and the enlargement scalpel for tunnel enlargement. The distance marker and guard are used for depth estimation, in some cases, the tunnel opening guide can be inserted in the soft tissue to guide the tunnel creation. The tunnel dilator will follow the tunnel opening guide to create the tunnel to the operative site. When the tunnel has been created, the visual enhancer end is inserted to enhance the surgical visual field. The surgeon can use a scalpel, scissors or other instrument as required passed through the tunnel created by the visual enhancer end. The associated lighting tool and lighting transmitter can be used as required to provide extra light on the surgical field.

### BACKGROUND OF THE INVENTION

The invention relates to a specialized instrument to enhance the visual field of a surgeon during a specific “minimally invasive surgery” procedure.

In modem surgery practice, it is preferred to use minimally invasive surgery when possible because of improved wound improvement, decreased wound infection, and a shorter recovery period. For example, a minimally invasive plates (MIPO) technique is now used worldwide in fractured bone fixation, because of rapid bone healing and less complications. However, minimally invasive surgery has some limitations in the visual field of the surgeon, necessitating the use of some blind techniques, which increase the risk of nerve transaction.

“The Carpal Tunnel Syndrome Release Operation” is one such new ‘minimally invasive surgery’ technique, in which a small incision is made into the wrist in order to release the nerve. The old technique made a longer incision, which of course required a longer healing time. The new technique has a certain limitation, with a limited visual field. and we thought that it would be possible to design to alleviate this problem—and the result was the Surgical Visual Field Enhancer.

Our aim in designing this instrument was to provide for surgeons an instrument, which would enhance their visual field during the carpal tunnel release operation, others minimally invasive surgery, and reduce the risk of nerve damage resulting from a poor visual field.

### SUMMARY OF THE INVENTION

The Surgical Visual Field Enhancer is used to improve the visual field under a limited skin incision. It includes various parts to make the surgeon's work easier, such as the removable handle, the tunnel opening guide, the tunnel dilator, the lighting tool and the lighting transmitter.

Initially, the user holds the surgical tool body of the surgical visual field enhancer and uses the tunnel opening end to create a tunnel through the soft tissue. The tunnel opening end is composed of the tunnel opening tip and the enlargement scalpel for tunnel enlargement. The distance marker and guard are used for depth estimation. In some cases, the tunnel opening guide can be inserted in the soft tissue to guide the tunnel creation. The tunnel dilator will follow the tunnel opening guide to create the tunnel to the operative site.

When the tunnel has been created, the visual enhancer end is inserted to enhance the surgical visual field. The surgeon can use a scalpel, scissors or other instrument as required passed through the tunnel created by the visual enhancer end. The associated lighting tool and lighting transmitter can be used as required to provide extra light on the surgical field.

References

Chapman et al. Complete median nerve transection as a complication of carpal tunnel release with a carpal tunnel tome. Am J Orthop (2001) vol. 30 (8) pp. 652-3

Cellocco et al. Mini-open blind procedure versus limited open technique for carpal tunnel release: a 30-month follow-up study. The Journal of hand surgery (2005) vol. 30 (3) pp. 493-9

Zhiquan et al Minimally invasive plating osteosynthesis (MIPO) of middle and distal third humeral shaft fractures. J Orthop Trauma (2007) vol. 21 (9) pp. 628-33

Apivatthakakul et al Minimally invasive plate osteosynthesis (MIPO) of the humeral shaft fracture. Is it possible? A cadaveric study and preliminary report. Injury (2005) vol. 36 (4) pp. 530-8

Sosef et al Minimal invasive fixation of proximal humeral fractures with an intramedullary nail: good results in elderly patients. Archives of orthopaedic and trauma surgery (2009) pp.

### BRIEF DESCRIPTION OF THE DRAWINGS

FIG. 1 is an orientation views of all components of the surgical visual field enhancer apparatus, showing the removable handle, the tunnel opening guide, the tunnel dilator, the lighting tool and the lighting transmitter.

FIG. 2 is a view of the palmar side of the hand inserting the tunnel opening end to create the tunnel.

FIG. 3 shows the insertion of the visual enhancer end to increase the surgical visual field.

FIG. 4 illustrates the different lengths of tunnel opening neck, which are available, and the two kinds of guard.

FIG. 5 shows the different tunnel opening tip shapes available.

FIG. 6a shows the different kinds of the tunnel opening guides available.

FIG. 6b illustrates the tunnel opening guide with the surgical field enhancer.

FIG. 7 gives examples of the different kinds of guiding hole that can be made.

FIG. 8 shows the different kinds of visual enhancer end.

FIG. 9a shows the surgical field enhancer before attachment of the lighting tool

FIG. 9b shows the attachment of the lighting tool to the surgical field enhancer.

FIG. 10a shows the surgical visual field enhancer before attachment of the lighting tool directly to the lighting transmitter's connector.

FIG. 10b shows the attachment of the lighting tool directly to the lighting transmitter's connector.

FIG. 11 are views of the different kinds of lighting transmitters which can be joined to the lighting tool.

FIG. 12a shows the attachment of the removable handle to the surgical field enhancer.

FIG. 12b showing how the removable handle is attached to the surgical field enhancer, insertion into a special slot.

FIG. 12c shows after attachment of the removable handle.

FIG. 12d another view after attachment of the removable handle to the visual enhancer end of the surgical field

FIG. 13a and B: views of the tunnel opening end with a permanent handle.

### COMPLETE DISCLOSERSURE OF INVENTION

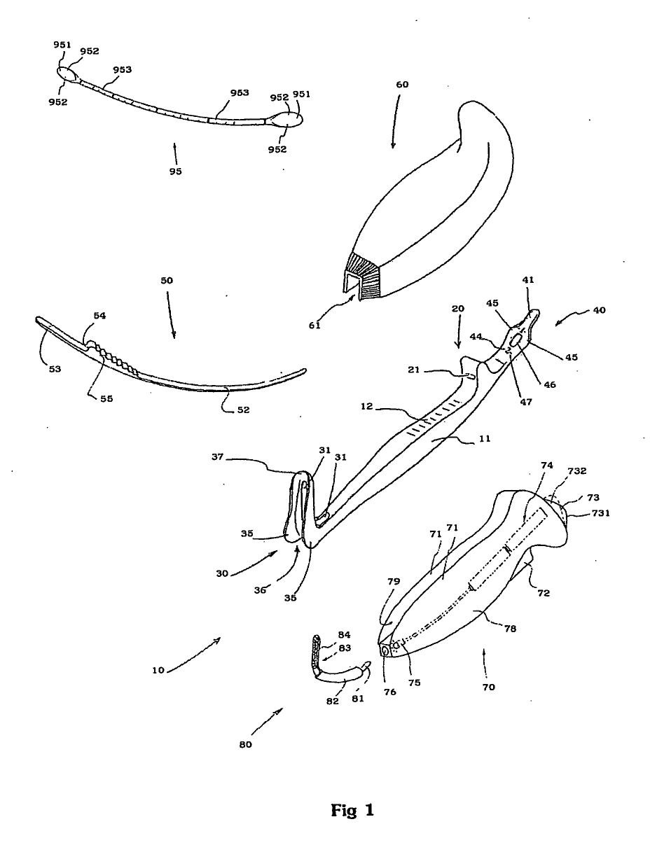

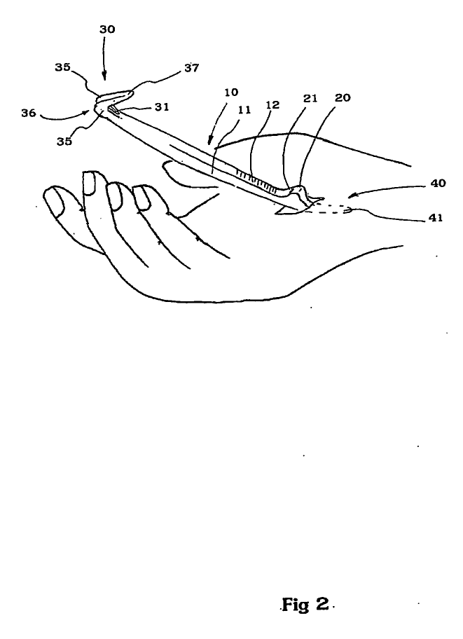

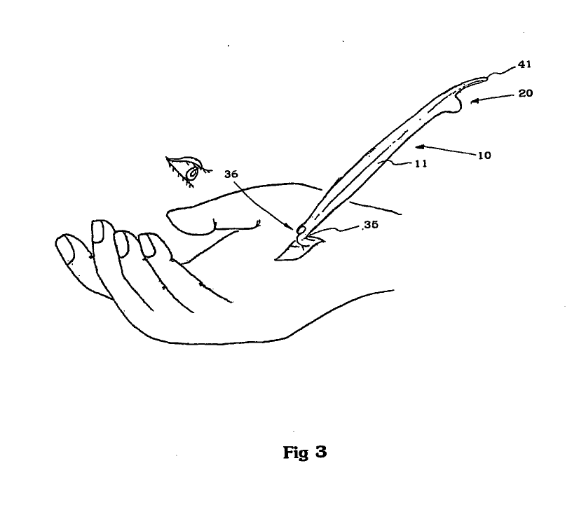

Referring now to the drawings, and more particularly to FIG. 1, the medical apparatus is composed of six tools, a surgical visual field enhancer 10, removable handle 60, a ‘tunnel opening’ guide 50, tunnel dilator 95, lighting tool 70 and lighting transmitter 80. As shown in FIG. 2 and FIG. 3, the surgical visual field enhancer 10 is the main tool to use for the two steps of surgery, which are the tunnel creation process and the visual enhancement process. The other parts of this device are additional tools, which can be applied in certain situations that will increase the convenience of usage.

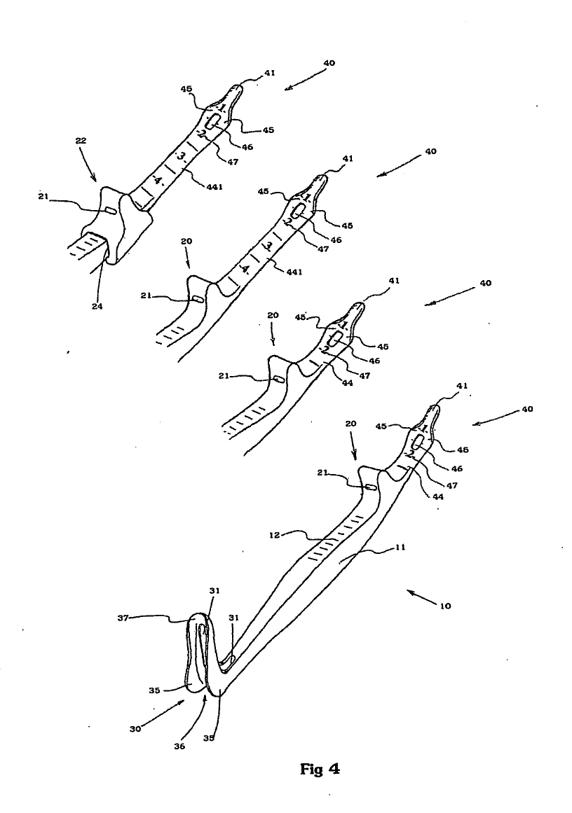

In FIG. 4, the surgical visual field enhancer 10 is diagramed, which has four major parts, the surgical tool body 11, the guard 20, the visual enhancer end 30 and the tunnel opening end 40. In the tunnel creation step, the tunnel opening end 40 is inserted into the soft tissue. The user holds the surgical tool body 11 with their thumb placed on the antigliding groove 12 for firm control during the tunnel creation process. The tunnel opening tip 41 is initially inserted, followed by the enlarging scalpel 45, which creates the tunnel by pushing the tool forward.

Still referring to FIG. 4, the tunnel opening end 40 has different sizes of tunnel opening neck 44. In cases where an extra long tunnel is needed, the long-neck 441 is used, with the addition of a guard 20 to monitor the tunnel depth. The adjustable guard 22 has an adjustable tunnel 24. The adjustable guard 22 slides on the tunnel opening long neck 441 to monitor and control the length of the tunnel using the length adjustable system. A distance marker 47 is calibrated on the tunnel opening device's neck 44 to allow accurate estimation of the depth of the tunnel.

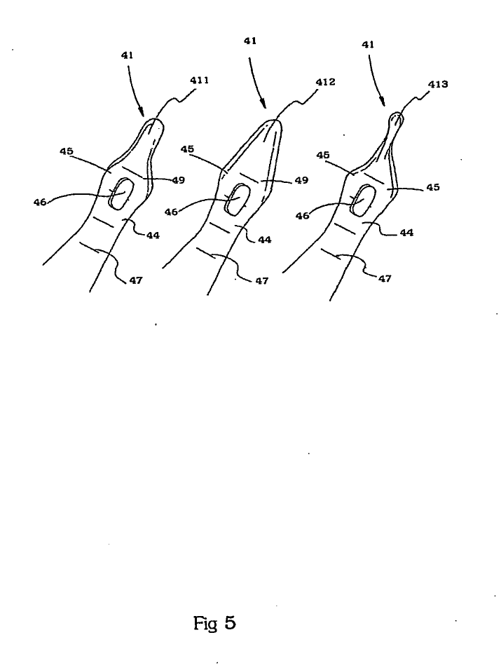

In FIG. 5, the different shapes of the tunnel opening tip 41 are shown, which can be used in different types of soft tissues. The small tunnel opening 411 has a small flat tiny tip, which is suitable for use in softer tissues, while the arrow type tunnel opening 412 has a tiny tip and wider slope at the bilateral base, which is suitable for use in harder tissues. The vertical type tunnel opening 413 has a tiny vertical tip and wider slope at the bilateral base which is suitable for use in a limited vertical entrance.

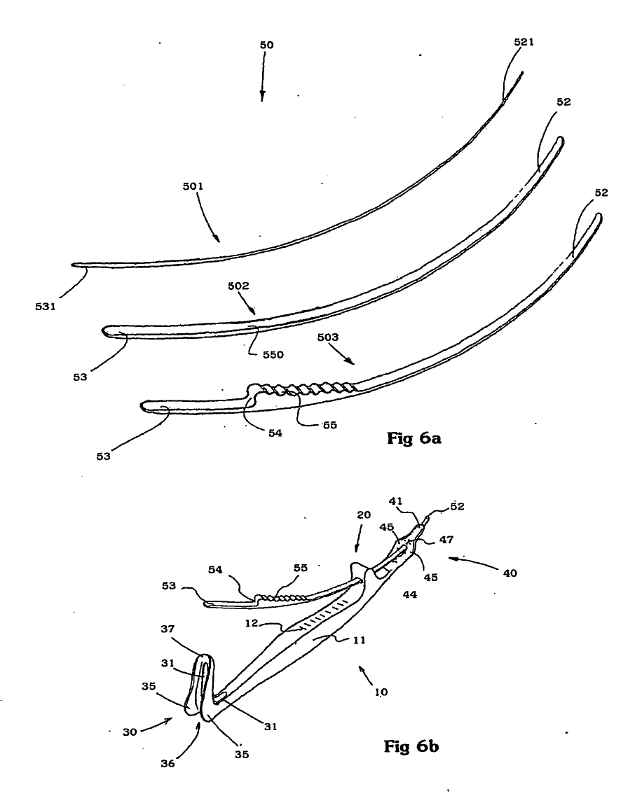

In FIG. 6a, a tunnel opening guide 50 is an additional tool to guide the direction in the process of tunnel creation. For insertion in different types of soft tissues, different sizes and shapes of tunnel opening guide 50 are available. The tunnel opening guide-wired version 501 has a small round tiny wire that is suitable for harder tissues. The simple tunnel opening guide 502 is a flat small shape that is suitable for softened tissues. The antislip tunnel opening guide 503 is a small flat shape with an antislip handle 55 and deepened guard stopper 54 that is suitable for general tissues such as subcutaneous tissues. The tunnel opening guide 50 has two ends, one being a tunnel guide starter 53 and the other being the body of the tunnel opening guide 52. For the tunnel opener guide-wired version 501, there are two small ends, which are a tunnel guide starter-wired version 531 and the body of the tunnel opening guide-wired version 521. The tunnel guide starter 53 is the initial part to be inserted that can control direction easily. The user can grip the handle and push the tunnel guide starter 53 forward for a controlled distance limited by the deep guard stopper 54. The handle has two types simple handle 550 or antislip handle 55. The simple handle 550 has smooth surface that is suitable for softer tissues. The antislip handle 55 has rough surface that is suitable for harder tissues. After starting with tunnel guide starter 53, the user passes the body of the tunnel opening guide 52 through the guiding hole of the guard 21 and then the guiding hole of the neck 46 as shown in FIG. 6b. Then the body of the tunnel opening guide 52 is inserted in the soft tissue. Under guidance, the tunnel opening end 40 can then create a tunnel following the direction of the tunnel opening guide 50.

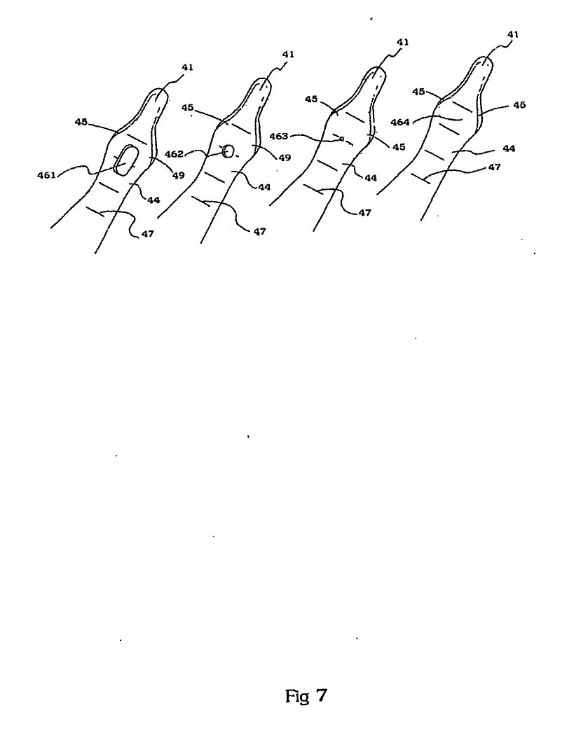

In FIG. 7, the different kinds of tunnel opening guide 50 suitable for insertion through different kinds of guiding holes of the neck 46 are shown. The standard type of guiding hole 461 has a big hole that is suitable for simple tunnel opening guide 502 and antislip tunnel opening guide 503. The small type of guiding hole 462 makes a smaller round hole that is suitable for the small tunnel opening guide-wired version 501. The pin type of guiding hole 463 has the smallest round hole that is suitable for the smallest size of tunnel opening guide-wired version 501. The holeless surface 464 has no hole on the neck, which is suitable for cases that do not need to use the tunnel opening guide 50.

Referring to FIG. 1, the tunnel dilator 95 is an additional tool for dilating a tunnel before the visual enhancement process. The tunnel dilator 95 is composed of a blunting tip 951, bilateral sharpened edges 952 and a handle with a marker 953. The rounded blunting tip 951 is used for the initial insertion zone. The bilateral sharpened edges 952 have wider edges for enlarging the tunnel. The handle with a marker 953 is a handling stick that has a marker for estimating depth.

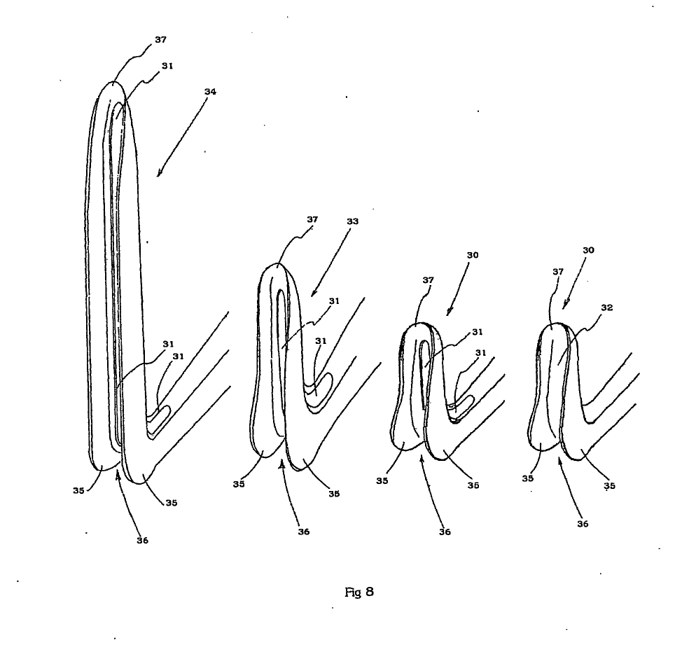

For the visual enhancement process, the visual enhancer end 30 is inserted into the soft tissue along the tunnel, which has been created by the tunnel opening 40. FIG. 8 shows the visual enhancer end 30, which consists of a small tip 37, a lateral soft tissue protector 35 and a visual space 36. The small tip 37 has a special small round end which is small enough for initial insertion, after which the bilateral plates of the lateral soft tissue protector 35 are passed through to protect the soft tissues. When the user passes the visual enhancer end 30 deeply through the soft tissues, a visual space 36 is created, a tunnel through which the user can see clearly along the way. The user can then pass other tools such as a surgical knife or scissors though the visual space 36. The visual space 36 can have different shapes and sizes depending on the depth and size of the tunnel. A large visual enhancer end 33 has a longer visual space 36 that can be used for deep tunnels. An extra large visual enhancer end 34 has the longest visual space 36 that is used for the deepest tunnels. The visual enhancer end 30 has a lighting slot 31, which is a special slot for inserting additional tools such as a lighting transmitter 80. In cases where the lighting transmitter 80 is not needed, a slot less surface 32 is available for the visual enhancer end 30.

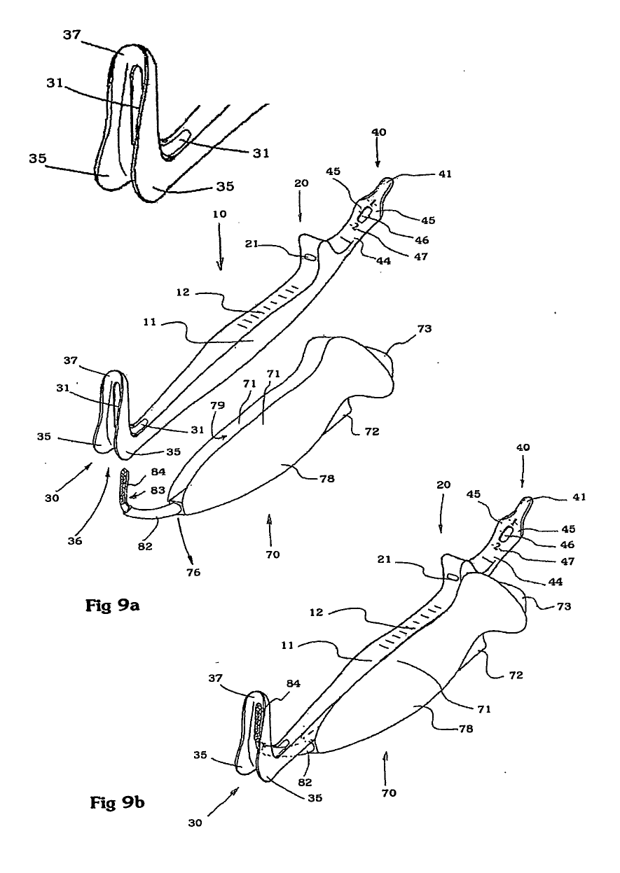

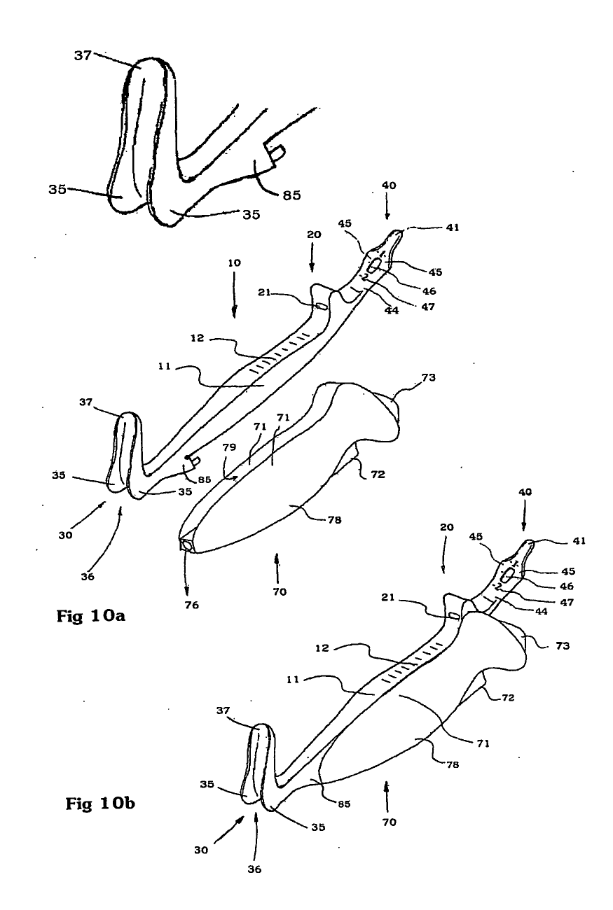

In FIG. 9a and FIG. 9b, the surgical visual field enhancer 10 can be attached to a lighting tool 70 and lighting transmitter 80 via the surgical tool socket 79. The lighting tip 83 is a small transparent tool that can be passed through the lighting slot 31 for lighting the visual space 36. The surgical tool body 11 is inserted into the surgical tool socket 79. In FIG. 10a and FIG. 10b, a special transparent version of surgical visual field enhancer 10 has a lighting transmitter's for visual enhancing 85 can be joined directly to the lighting tool 70 for lighting the visual space 36.

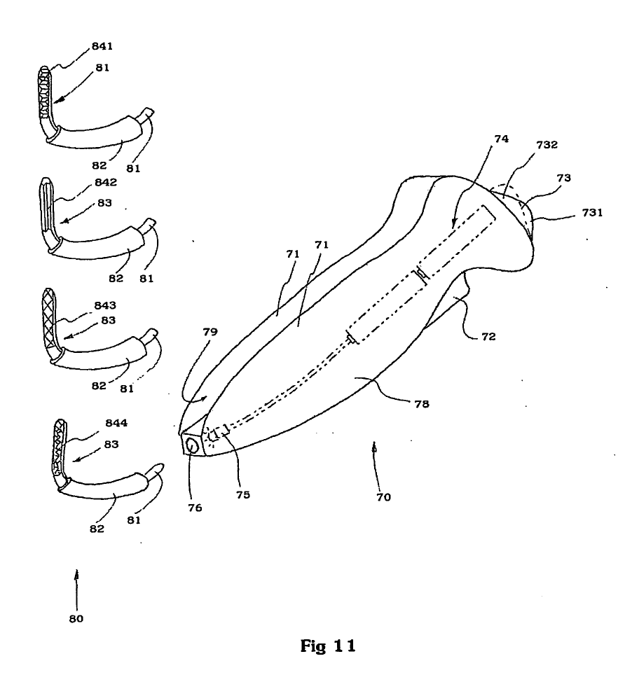

FIG. 11: the lighting tool 70 is composed of a lighting box 78, a locking fin 71, a lighting button 72, an on-off switch 73, a lighting transmitter slot 76 and a surgical tool socket 79. The lighting box 78 contains the power source 74 and lighting source 75. The lighting button 72 is a special button that is pressed to give light while the surgical tool body 11 is being used. In cases where the light is needed for a protracted period of time, the user can turn the light on and off with the switches 731 and 732. The lighting transmitter slot 76 is a special slot for joining the lighting transmitter's connector 81. The lighting transmitter 80 has many designs for transmitting light from the lighting resource 75. The lighting transmitter 80 is composed of a lighting transmitter connector 81, a lighting saver 82, a lighting tip 83 and a lighting surface 84. A lighting saver 82 is a kind of reflecting light material which covers or coats the lighting transmitter 80. A lighting surface 84 has differences kinds of surface pattern such as a cross lighting surface 841, a longitudinal lighting surface 842, a diamond shape lighting surface 843 and a random lighting surface 844.

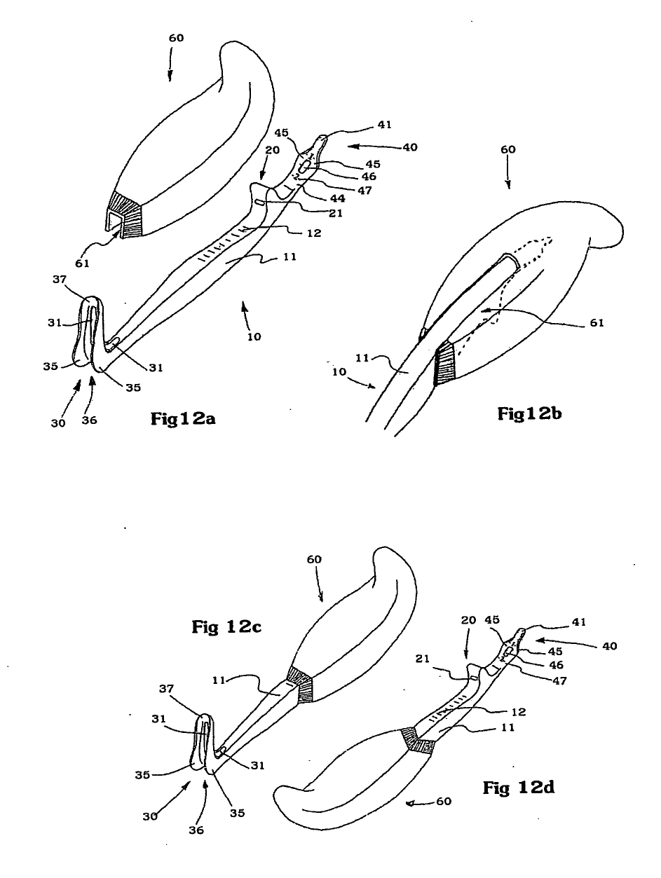

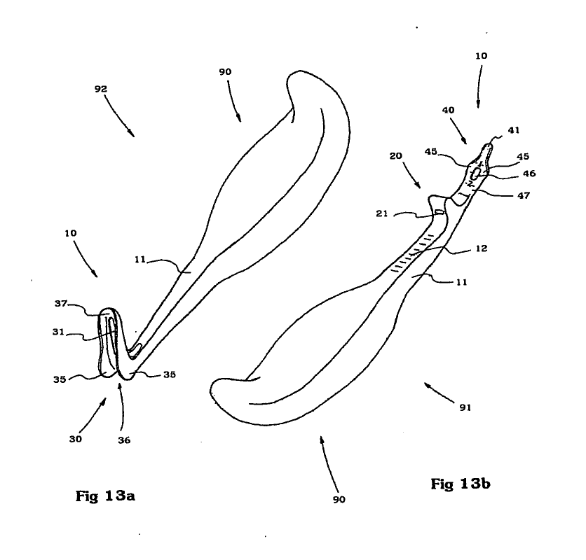

FIG. 12a shows a removable handle 60, which is an additional tool for improving the holding power. In FIG. 12b, shows a handle slot 61, which is inserted the surgical visual field enhancer 10 inside firmly. In FIG. 12c and FIG. 12d, we see the surgical visual field enhancer 10 can have attached a removable handle 60 at either end for convenience in handling any sides, tunnel opening end 40 or the surgical visual field enhancer 10. In FIG. 13a and FIG. 13b, a surgical visual field enhancer 10 can also be designed to be fused with a permanently designed handle 90. There are a tunnel opening end with permanent designed handle 91 and a visual enhancer end with permanent designed handle 92.

1-29. (canceled)

30. A surgical visual field enhancer, comprising:
an elongated body;
a tunnel opening end about a first end of the elongated body; and
a visual enhancer end about a second end opposite the first end of the elongated body, the visual enhancer end having a visual enhancer extending substantially transverse to the elongated body, wherein the visual enhancer includes:
lateral protectors extending from the visual enhancer; and
a visualizing space formed between the lateral protectors.

31. The surgical visual field enhancer of claim 30, further comprising a guard extending substantially transverse from the elongated body between the tunnel opening end and the visual enhancer end.

32. The surgical visual field enhancer of claim 31, wherein the guard is movable along the elongated body.

33. The surgical visual field enhancer of claim 30, further comprising a handle attachable to the elongated body.

34. The surgical visual field enhancer of claim 30, further comprising a lighting tool attachable to the elongated body for illuminating the visualizing space.

35. The surgical visual field enhancer of claim 30, wherein the tunnel opening end is tapered.

36. The surgical visual field enhancer of claim 30, wherein the tunnel opening end includes:
a tip; and
enlargement scalpels extending from the tip.

37. The surgical visual field enhancer of claim 36, wherein the tunnel opening end further includes a marker or numbering for estimating length.

38. The surgical visual field enhancer of claim 30, wherein the visual enhancer further comprises a slot.

39. The surgical visual field enhancer of claim 30, wherein the elongated body includes an antigliding groove.

40. A surgical system for enhancing the visual field of a surgical site comprising:
an elongated surgical visual field enhancer that includes a tunnel opening end having:
a tip extending in a first direction for insertion into the surgical site; and
a guiding hole spaced from the tip; and
a tunnel opening guide insertable through the guiding hole for extending substantially in the first direction.

41. The surgical system of claim 40, further comprising:
a visual enhancer end opposite the tunnel opening end, the visual enhancer end having a visual enhancer extending substantially transverse to a main body portion of the elongated surgical visual field enhancer, wherein the visual enhancer includes:
lateral protectors extending from the visual enhancer; and
a visualizing space formed between the lateral protectors.

42. The surgical system of claim 41, further comprising a lighting tool attachable to the surgical visual field enhancer for illuminating the visualizing space.

43. The surgical system of claim 42, wherein the lighting tool comprises a tool socket for receiving the elongated surgical visual field enhancer.

44. The surgical system of claim 41, wherein the visual enhancer further comprises a slot.

45. The surgical system of claim 44, further comprising a lighting tool having a lighting tip configured to be received within the slot and attachable to the surgical visual field enhancer for illuminating the visualizing space.

46. The surgical system of claim 40, wherein the tunnel opening end is tapered.

47. The surgical system of claim 40, wherein the tunnel opening end further includes enlargement scalpels extending from the tip.

48. The surgical system of claim 40, wherein the surgical visual field enhancer further comprises a guard for limiting a depth of insertion of the tunnel opening end within the surgical site.

49. The surgical system of claim 48, wherein the guard includes a guard guiding hole for receiving the tunnel opening guide.

50. The surgical system of claim 48, wherein the guard is movable along the elongated surgical visual field enhancer.

51. The surgical system of claim 40, further comprising a handle attachable to the surgical visual field enhancer.

52. The surgical system of claim 40, wherein the tunnel opening guide comprises a guard stopper.

53. The surgical system of claim 40, wherein the tunnel opening end further includes a marker or numbering for estimating length.

54. The surgical system of claim 40, wherein the elongated surgical visual field enhancer includes an antigliding groove.

55. A surgical kit for enhancing the visual field of a surgical site comprising:
the surgical system of claim 40; and
a tunnel dilator.
